(19) United States
(12) Patent Application Publication
(10) Pub. No.: US 2015/0343036 A1
(43) Pub. Date: Dec. 3, 2015

(54) METHODS FOR REDUCING THE OCCURRENCE OR PREVENTING FORMATION OF BLADDER CALCULI

(71) Applicant: Allergan, Inc., Irvine, CA (US)

(72) Inventors: Christopher A. Dadas, Strongsville, OH (US); Tamer Aboushwareb, Kernersville, NC (US)

(21) Appl. No.: 14/722,473

(22) Filed: May 27, 2015

Related U.S. Application Data
(60) Provisional application No. 62/003,941, filed on May 28, 2014.

(51) Int. Cl. A61K 38/48

(52) U.S. Cl. CPC A61K 38/4893; C12Y 304/24069

(57) ABSTRACT

Methods for reducing the occurrence or preventing formation of bladder calculi associated with bladder augmentation or bladder reconstruction.

### CROSS-REFERENCES TO RELATED APPLICATIONS

This application claims the benefit of U.S. Provisional Application Ser. No. 62/003,941, filed May 28, 2014, which is incorporated herein by reference in its entirety.

### FIELD

The present disclosure relates to methods for reducing the occurrence or preventing the formation of calculi. In particular, the present disclosure relates to methods for protecting against bladder augmentation-associated complications by reducing the occurrence of or preventing the formation of calculi using neurotoxins.

### BACKGROUND

Bladder calculi are stones or calcified materials that are present in the bladder, usually when the urine becomes concentrated. Medical conditions, trauma or injuries that result in urinary incontinence, an enlarged prostate, or recurring urinary tract infections are common causes of concentrated and stagnant urine. Symptoms of bladder calculi may include abdominal pain, pain when urinating or blood in urine. A tiny bladder stone may pass out of the body on its own. However, removal of larger ones requires medical intervention. Untreated bladder calculi can eventually result in infections and complications. Increased calculi formation has been observed with bladder augmentation or bladder reconstruction surgical procedures.

Bladder augmentation, also called augmentation cystoplasty, is a surgical procedure to increase the size of the bladder. The bladder can be enlarged, or it can be partially or completely replaced. Bladder augmentation, reconstruction, or diversion procedures are performed on patients who lack adequate bladder capacity, high detrusor pressures, or have failed less invasive therapies for detrusor over activity. Bladder reconstruction has also been routinely used after radical cystectomy surgery due to malignant bladder tumors. Augmentation cystoplasty or complete bladder reconstruction involves enlarging the bladder by patching a non-bladder tissue, such as a detached segment of intestine (bowel) or stomach tissue, to the bladder to expand the bladder capacity or create a continent diversion (FIG. 1).

Methods employed to decrease bladder calculi formation include regular bladder irrigation and Clean Intermittent Catherization (CIC). However, such treatments are not always efficient. Other methods of calculi removal include ESWL (extracorporeal shock wave lithotripsy), cystoscopic lithotripsy and removal, but recurrence of these complications often ensues.

Thus, there is a need for alternative treatment methods that reduce the occurrence or preventing the formation of calculi associated with bladder augmentation or bladder reconstruction.

### SUMMARY OF THE INVENTION

Aspects of the present disclosure provide a method for reducing the occurrence or preventing the formation of calculi associated with a bladder surgical procedure in a patient in need thereof. In some embodiments, the present method alleviates the pain, discomfort, inflammation associated with bladder calculi formation following bladder augmentation or reconstruction. In one aspect, the present method comprises locally administering a clostridial derivative to a patient in need of bladder augmentation or reconstruction. In some embodiments, the clostridial derivative is a native or recombinant neurotoxin, a recombinant modified toxin, fragments thereof, a Targeted vesicular Exocytosis Modulator (TEM), or combinations thereof. In one embodiment, the present method comprises locally administering a clostridial derivative into a section of the non-bladder tissue to be grafted to the bladder of a patient. In one embodiment, the non-bladder tissue to be grafted is an intestine tissue. In some other embodiments, the non-bladder tissue to be grafted is a stomach tissue.

In one embodiment, the present disclosure provides a method for alleviating at least one symptom associated with bladder augmentation or reconstruction in a patient in need thereof, the method comprising locally administering a therapeutically effective amount of a clostridial derivative to a section of the non-bladder tissue to be grafted to the bladder of the patient, thereby alleviating the at least one symptom. In one embodiment, the clostridial derivative is administered to the non-bladder graft tissue before a bladder augmentation surgical procedure. In another embodiment, the clostridial derivative is administered during the surgical procedure. In another embodiment, the clostridial derivative is administered after the surgical procedure.

In another aspect, by alleviating one or more symptoms associated with bladder augmentation or reconstruction in a patient in need thereof, the present method provides functional improvement and thus improves the quality of life for the patient.

### BRIEF DESCRIPTION OF THE DRAWINGS

The following drawings are presented to illustrate aspects and features of embodiments of the present invention.

FIG. 1 is a diagram of an exemplary bladder augmentation procedure.

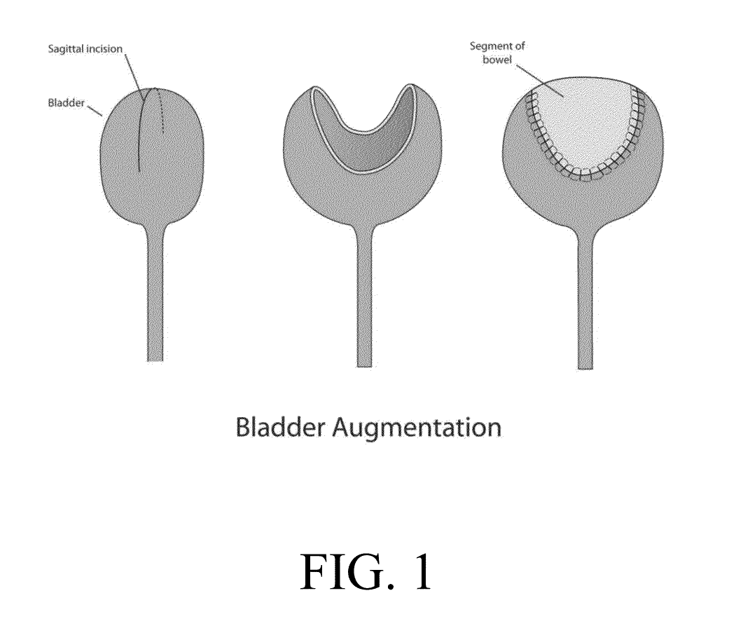

### DESCRIPTION OF THE PREFERRED EMBODIMENTS

Botulinum neurotoxins (BoNTs) such as, for example, BoNT/A, BoNT/B, etc., act on the nervous system by blocking the release of neurosecretory substances such as neurotransmitters. The action of BoNT is initiated by its binding to a receptor molecule on the cell surface, and then the toxin-receptor complex undergoes endocytosis. Once inside the cell, BoNT cleaves exocytotic specific proteins responsible for neurotransmitter docking and release from the cell known as the SNARE proteins (soluble N-ethylmaleimide-sensitive factor attachment protein receptor). The resulting transient chemodenervation has been utilized medically to block motor neurotransmission at the neuromuscular junction leading to a variety of therapeutic applications.

The following definitions apply herein:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a botulinum toxin to a subject, or alternatively a subject receiving a pharmaceutical composition. The present method can be performed via administration routes including intramuscular, non-intramuscular, intradermal, subcutaneous administration, intrathecal administration, intraperitoneal administration, implantation (for example, of a slow-release device such as polymeric implant or miniosmotic pump), instillation, or combinations thereof

“Alleviating” means a reduction in the occurrence of a pain or other symptoms associated with bladder calculi. Thus, alleviating includes some reduction, significant reduction, near total reduction, and total reduction. An alleviating effect may not appear clinically for between 1 to 7 days after administration of a clostridial derivative to a patient or sometimes thereafter.

“Botulinum toxin” means a neurotoxin produced by Clostridium botulinum, as well as a botulinum toxin (or the light chain or the heavy chain thereof) made recombinantly by a non-Clostridial species. The term “botulinum toxin”, as used herein, encompasses the botulinum toxin serotypes A, B, C, D, E, F and G, and their subtypes and any other types of subtypes thereof, or any re-engineered proteins, analogs, derivatives, homologs, parts, sub-parts, variants, or versions, in each case, of any of the foregoing. “Botulinum toxin”, as used herein, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins.

“Clostridial derivative” refers to a molecule which contains any part of a clostridial toxin. As used herein, the term “clostridial derivative” encompasses native or recombinant neurotoxins, recombinant modified toxins, fragments thereof, a Targeted vesicular Exocytosis Modulator (TEM), or combinations thereof.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate.

“Effective amount” as applied to the biologically active ingredient means that amount of the ingredient which is generally sufficient to induce a desired change in the subject. For example, where the desired effect is a reduction in calculi formation, an effective amount of the ingredient is that amount which causes at least a substantial reduction of bladder calculi and associated symptoms, and without resulting in significant toxicity.

“Implant” means a controlled release (e.g., pulsatile or continuous) composition or drug delivery system. The implant can be, for example, injected, inserted or implanted into a human body.

“Local administration” means administration of a pharmaceutical agent to or to the vicinity of a muscle or a subdermal location in a patient by a non-systemic route. Thus, local administration excludes systemic routes of administration, such as intravenous or oral administration.

“Peripheral administration” means administration to a location away from a symptomatic location, as opposed to a local administration.

“TEMs”, abbreviated for Targeted Exocytosis Modulators are retargeted endopeptidases that direct the catalytic activity of the light chain to specific types of neuronal cells or to target cells that were not affected by botulinum toxins expanding the beneficial clinical effect of inhibition of exocytosis in several human diseases.

“Treating” or “treatment” means to alleviate (or to eliminate) at least one symptom (such as, for example, hip and groin pain), either temporarily or permanently.

“Therapeutically effective amount” refers to an amount sufficient to achieve a desired therapeutic effect.

Aspects of the present disclosure provide in part a method for reducing the occurrence or preventing the formation of bladder calculi in a patient in need of bladder augmentation or reconstruction, the method comprising locally administering a composition comprising a therapeutically effective amount of a clostridial derivative to a non-bladder tissue to be grafted to the bladder.

Without wishing to be bound by theory, a physiological mechanism can be set forth to explain the efficacy of the present disclosure. The increased bladder calculi formation following bladder augmentation or reconstruction may be attributed to mucus production from the non-bladder tissue which is grafted onto the bladder to increase its size. Once grafted onto the bladder, the non-bladder tissue still maintains its secretory function. For example, a stomach tissue continues secreting digestive juices, a bowel or intestine tissue continues secreting mucus. The mucus production by the non-bladder tissue may prevent efficient urine drainage, promote bacterial growth, and alter metabolism and absorption of materials within the bladder, which leads to increased calculi formation. Mucus secretion from mucus secreting cells is mediated cholinergically (i.e. via neurotransmitters, including acetylcholine). Thus, administration of a composition comprising a clostridial derivative, for example a botulinum toxin, to the non-bladder graft tissue blocks or decreases the frequency of acetylcholine release which in turns reduces or blocks cholinergic controlled mucus secretions from the non-bladder graft tissue. The reduced or blocked mucus secretion from the non-bladder graft tissue will lead to a reduction in occurrence of bladder calculi following a bladder augmentation or reconstruction procedure.

In some embodiments, the composition is administered by injections, including intramuscular injections or non-intramuscular injections. In alternative embodiments, the composition is administered by instillation.

In some embodiments, the present method comprises administering the composition comprising the clostridial derivative to the enlarged bladder. In some embodiments, the composition is administered to the bladder wall. In other embodiments, the composition is administered to the bladder neck. In some embodiments, the composition is administered to the non-bladder graft tissue.

In some embodiments, the clostridial derivative includes a native, recombinant clostridial toxin, recombinant modified toxin, fragments thereof, TEMs, or combinations thereof. In some embodiments, the clostridial derivative is a botulinum toxin. In some embodiments, the botulinum toxin can be a botulinum toxin type A, type B, type C₁, type D, type E, type F, or type G, or any combination thereof. The botulinum neurotoxin can be a recombinantly made botulinum neurotoxins, such as botulinum toxins produced by E. coli. In alternative embodiments, the clostridial derivative is a TEM.

In some embodiments, the botulinum neurotoxin can be a modified neurotoxin, that is a botulinum neurotoxin which has at least one of its amino acids deleted, modified or replaced, as compared to a native toxin, or the modified botulinum neurotoxin can be a recombinant produced botulinum neurotoxin or a derivative or fragment thereof. In certain embodiments, the modified toxin has an altered cell targeting capability for a neuronal or non-neuronal cell of interest. This altered capability is achieved by replacing the naturally-occurring targeting domain of a botulinum toxin with a targeting domain showing a selective binding activity for a non-botulinum toxin receptor present in a non-botulinum toxin target cell. Such modifications to a targeting domain result in a modified toxin that is able to selectively bind to a non-botulinum toxin receptor (target receptor) present on a non-botulinum toxin target cell (re-targeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

The clostridial derivative, such as a botulinum toxin, for use according to the present invention can be stored in lyophilized, vacuum dried form in containers under vacuum pressure or as stable liquids. Prior to lyophilization the botulinum toxin can be combined with pharmaceutically acceptable excipients, stabilizers and/or carriers, such as, for example, albumin, or the like. In embodiments containing albumin, the albumin can be, for example, human serum albumin, or the like. The lyophilized material can be reconstituted with a suitable liquid such as, for example, saline, water, or the like to create a solution or composition containing the botulinum toxin to be administered to the patient.

In some embodiments, the clostridial derivative is provided in a controlled release system comprising a polymeric matrix encapsulating the clostridial derivative, wherein fractional amount of the clostridial derivative is released from the polymeric matrix over a prolonged period of time in a controlled manner. Controlled release neurotoxin systems have been disclosed for example in U.S. Pat. Nos. 6,585,993; 6,585,993; 6,306,423 and 6,312,708, each of which is hereby incorporated by reference in its entirety.

The therapeutically effective amount of the clostiridial derivative, for example a botulinum toxin, administered according to the present method can vary according to the potency of the toxin and particular characteristics of the pain being treated, including its severity and other various patient variables including size, weight, age, and responsiveness to therapy. The potency of the toxin is expressed as a multiple of the LD₅₀ value for the mouse, one unit (U) of toxin being defined as being the equivalent amount of toxin that kills 50% of a group of 18 to 20 female Swiss-Webster mice, weighing about 20 grams each.

The therapeutically effective amount of the botulinum toxin, in the present method can vary according to the potency of a particular botulinum toxin, as commercially available Botulinum toxin formulations do not have equivalent potency units. For example, one unit of BOTOX® (onabotulinumA), a botulinum toxin type A available from Allergan, Inc., has a potency unit that is approximately equal to 3 to 5 units of DYSPORT® (abobotulinumA), also a botulinum toxin type A available from Ipsen Pharmaceuticals. MYOBLOC®, a botulinum toxin type B available from Elan, has a much lower potency unit relative to BOTOX®. In some embodiments, the botulinum neurotoxin can be a pure toxin, devoid of complexing proteins, such as XEOMIN® (incobotulinumtoxinA). One unit of IncobotulinumtoxinA has a potency approximately equivalent to one unit of onabotulinumtoxinA. Thus, the quantity of toxin administered and the frequency of its administration will be at the discretion of the physician responsible for the treatment and will be commensurate with questions of safety and the effects produced by a particular toxin formulation.

The dosages used in human therapeutic applications are roughly proportional to the mass of the tissue being injected. Typically, the dose of a clostridial derivative administered to the patient may be up from about 0.01 to about 1,000 units; for example, up to about 500 units, and preferably in the range from about 80 to about 460 units per patient per treatment, although smaller of larger doses may be administered in appropriate circumstances.

In some embodiments, the present method comprises administering a composition comprising about 10-500 units of a botulinum toxin type A, such as BOTOX®, into the non-bladder graft tissue. In some embodiments, the present method comprises administering a composition comprising about 25-300 units of BOTOX® into the graft tissue. In one specific embodiment, the present method comprises administering a composition comprising about 10-500 units of BOTOX® by instillation into the enlarged bladder. In some embodiments, the composition is administered to the bladder or its vicinity, e.g. the detrusor. In certain embodiments, the dosage can range from about 10 Units to about 200 U per treatment. In some embodiments, the pharmaceutical composition can be administered at multiple sites, ranging from 1 site up to about 50 sites. In some embodiments, if the neurotoxin is botulinum toxin type B, the dosage is approximately 50 times greater than the functionally equivalent dosage of botulinum toxin type A. In some embodiments, the administering is by injections to the bladder, the non-bladder graft tissue, or both. In alternative embodiments, the administering is by instillation.

In one embodiment, the composition comprising the clostridial derivative is administered before the bladder augmentation surgical procedure. In another embodiment, the composition comprising the clostridial derivative is administered during the surgical procedure. In another embodiment, the composition comprising the clostridial derivative is administered after the surgical procedure. In another embodiment, administration of the composition comprising the clostridial derivative is repeated periodically after the surgical procedure to minimize mucus secretion from the graft tissue to reduce the occurrence or prevent the formation of bladder calculi.

The treatment effects of the clostridial derivative can persist for between about 1 month and 5 years. Administration can be repeated as necessary. As a general guideline, botulinum toxin type A administered into or near muscle tissue has been observed to produce flaccid paralysis at target site muscles for up to about 3 to 6 months. However, increased efficacy of the treatment using botulinum toxin type A is expected to happen when the toxin is administered according to the disclosed method at about 3 month intervals. Repeated administration of the clostridial derivative according to aspects of the present method minimizes mucus secretion of the non-bladder graft tissue and therefore reduces the occurrence or prevents formation of bladder calculi.

In another aspect, the present disclosure provides a method for alleviating the pain, discomfort and inflammation due to bladder calculi in a patient who had bladder augmentation or reconstruction, the method comprising locally administering a composition comprising a therapeutically effective amount of a clostridial derivative into the enlarged bladder.

A method within the scope of the present disclosure can provide improved patient function. “Improved patient function” can be defined as an improvement measured by factors such as a reduced pain, increased ambulation, healthier attitude, more varied lifestyle and/or healing permitted by normal muscle tone and function. Improved patient function is may be measured with an improved quality of life (QOL) or Health-Related Quality of Life (HRQL). Scores obtained can be compared to published values available for various general and patient populations.

The following non-limiting examples provide those of ordinary skill in the art with specific preferred methods to treat bladder calculi formation associated with bladder augmentation or bladder reconstruction procedures within the scope of the present disclosure, and it is not intended to limit the scope of the invention. In the following examples various modes of non-systemic administration of a botulinum neurotoxin can be carried out. For example, by intramuscular injection, non-intramuscular injection, instillation or by implantation of a controlled release implant.

### EXAMPLES

The following non-limiting examples provide those of ordinary skill in the art with specific preferred methods to treat conditions within the scope of embodiments of the present invention and are not intended to limit the scope of the invention.

### Example 1—Botulinum Toxin Type A Therapy for Bladder Calculi Prevention

A 26-year-old patient is referred to the urologic clinic for a urodynamic evaluation from his physician. The patient reports experiencing frequent incontinence and high bladder pressure. He has a past history of a complete spinal cord injury at T-2 level of the spinal column 6 months previously. He has tried numerous oral anticholinergic medications, even at high doses to control his incontinence; which however had limited efficacy and which had several side effects including severe dry mouth and constipation. He has visited a urologist who recommended Botox treatment for his neurogenic incontinence and high bladder pressure to prevent renal damage. The patient however has limited access to the urologist due to his rural living situation. After a review of available options with his healthcare team, the patient has decided to proceed with a bladder augmentation surgery where a section of the gastrointestinal tract is used to add volume to his bladder to prevent potential kidney damage. During the bladder augmentation surgery, 100 units of Botulinum toxin type A are injected into a section of gastrointestinal tissue to prevent mucus secretion and development of bladder calculi.

### Example 2—Botulinum Toxin Type A Therapy to Reduce the Occurrence of Bladder Calculi

A 65-year-old male patient had a bladder augmentation surgical procedure due to high bladder pressure. A few months after the surgical procedure, he report severe abdominal pain, pain when he urinates and blood in his urine. He is diagnosed with large bladder calculi. The calculi are removed by open suprapublic systotomy. However, a year later, the same symptoms recur due to bladder calculi formation. After removal of the calculi, his urologist recommend administration of a botulinum toxin to the bladder, wherein 200 units of a botulinum toxin type A are administered at multiple sites to the bladder, including the non-bladder tissue that was grafted during the bladder augmentation procedure. The botulinum toxin treatment is repeated after 6 months. The patient remains free of bladder calculi for several months afterwards.

### Example 3

The method of Examples 1 and 2 is repeated, except that about 10,000 units of a botulinum toxin type B are administered.

### Example 4

The method of Examples 1 and 2 is repeated, except that 0.1 to 1000 micrograms (μg) of a TEM (Targeted Exocytosis Modulators) is administered to the bladder.

### Example 5

The method of Examples 1 and 2 is repeated, except the administration is by instillation to the bladder of the patient.

Many alterations and modifications may be made by those having ordinary skill in the art, without departing from the spirit and scope of the disclosure. Therefore, it must be understood that the described embodiments have been set forth only for the purposes of examples, and that the embodiments should not be taken as limiting the scope of the following claims. The following claims are, therefore, to be read to include not only the combination of elements which are literally set forth, but all equivalent elements for performing substantially the same function in substantially the same way to obtain substantially the same result. The claims are thus to be understood to include those that have been described above, those that are conceptually equivalent, and those that incorporate the ideas of the disclosure.

We claim:

1. A method for reducing the occurrence of bladder calculi in a patient in need of a bladder augmentation or bladder reconstruction surgical procedure, the method comprising locally administering a composition comprising a therapeutically effective amount of a clostridial derivative to the patient.

2. The method of claim 1, whether the administering is to a non-bladder tissue to be grafted to the bladder of the patient.

3. The method of claim 1, wherein the administering is to the bladder neck of the patient.

4. The method of claim 1, wherein the administering is before the bladder augmentation or bladder reconstruction surgical procedure.

5. The method of claim 1, wherein the administering is during the bladder augmentation or bladder reconstruction surgical procedure.

6. The method of claim 1, wherein the administering is after the bladder augmentation or bladder reconstruction surgical procedure.

7. The method of claim 1, wherein the clostridial derivative is a botulinum toxin.

8. The method of claim 7, wherein the botulinum toxin is selected from the group consisting of botulinuin toxin types A, B, C, D, E, F and G.

9. The method of claim 8, wherein the botulinum neurotoxin is type A.

10. The method of claim 9, wherein the therapeutically effective amount is between about 10 Units to about 500 U.

11. The method of claim 1, wherein the administering is by injection.

12. The method of claim 1, wherein the administering is by instillation.
